(19) United States
(12) Patent Application Publication
(10) Pub. No.: US 2026/0224280 A1
(43) Pub. Date: Aug. 6, 2026

(54) Electrode Probe For Radiofrequency Ablation

(71) Applicant: Stryker Corporation, Portage, MI (US)

(72) Inventors: Zachary Markley, Kalamazoo, MI (US); Camille Mercier, Kalamazoo, MI (US); Charles Jiral, Hamilton, MI (US); Ty N. McNall, Richland, MI (US)

(73) Assignee: Stryker Corporation, Portage, MI (US)

(21) Appl. No.: 19/128,792

(22) PCT Filed: Nov. 8, 2023
(86) PCT No.: PCT/US2023/036995

§ 371 Date: May 9, 2025

Related U.S. Application Data
(60) Provisional application No. 63/424,539, filed on Nov. 11, 2022.

(51) Int. Cl. A61B 18/14; A61B 18/00

(52) U.S. Cl. CPC A61B 18/1492; A61B 2018/00011; A61B 2018/00077; A61B 2018/00178; A61B 2018/00577; A61B 2018/1405; A61B 2218/002

(57) ABSTRACT

An electrode probe for radiofrequency ablation. A shaft extends from a hub and defines at least one lumen. A lead is disposed within the lumen, and at least one emitter is coupled to the lead. The emitter(s) are coupled onto a flexible distal portion of the shaft, such as through a swaging operation. The emitter(s) include a slotted portion defining slots sized for the conductive material to be deformed with swaging of the emitter onto the flexible distal portion. The emitter(s) may include a slotted portion and end portions, and the lead may be secured to one of the end portions through a laser welding operation. The slots may have a slot pitch within a range of 0.150 to 0.400 millimeters. The slots may be circumferentially disposed with a cut angle within a range of 50 to 90 degrees. Methods of manufacturing the electrode probe are also disclosed.

### PRIORITY CLAIM

This application claims priority to and all the benefits of U.S. Provisional Application No. 63/424,539, filed Nov. 11, 2022, the entire contents of which are hereby incorporated by reference.

### BACKGROUND

An ablation system directs energy to heat and destroy cells of problematic tissue. One example is the ablation of nerve tissue to cease transmitting pain signals to the brain. Another example includes the ablation of tumors of the liver, kidney, lung, and bone. When the pathology is intraosseous, for example, a bone tumor, an introducer assembly may facilitate positioning an electrode probe at a target location within the bone. In certain instances, it may be desirable for the introducer assembly to provide curved access difficult anatomical locations. One example includes accessing a bone tumor positioned posteriorly or contralateral within a vertebral body of the spine, and another example includes accessing the trunk of the basivertebral nerve. Many known electrode probes, particularly those with infusion or cooling capabilities, are incapable of flexing sufficiently follow the curve of the introducer assembly without compromise to its function. Moreover, the construction of many known electrode probes is intricate and thus associated with increased cost of manufacturing and assembly and increased potential risk of component failure. Therefore, there is a need in the art for an electrode probe for an ablation system that overcomes one or more of the aforementioned disadvantages.

### SUMMARY

The present disclosure is directed to an an ablation system and an electrode probe for radiofrequency (RF) ablation of tissue, and a method of manufacturing the same. The ablation system includes a console, the electrode probe, and optionally an infusion module. The console includes a source of RF energy, and optionally a display providing a user interface.

The electrode probe includes a handle or hub, and a shaft coupled to and extending from the hub. The hub may include a power coupler configured to be removably coupled with a power line. The power line may also transmit data between the console and the electrode probe. The hub includes a fluid coupler configured to be arranged in fluid communication with the infusion module. The shaft may include a flexible elongate body forming at least a flexible distal portion of the shaft. The shaft may also include a proximal portion, which may be flexible or rigid. A rigid sleeve may be coaxially overlying a portion of the elongate body. The elongate body may be polymeric, in other words, at least partially formed from a polymer. The extrusion of the elongate body provides one or more lumens and/or one or more grooves.

The emitters are flexible in addition to the flexibility of the elongate body. The proximal and distal emitters coupled to the flexible distal portion of the elongate body. The emitters may be secured to the flexible distal portion through a crimping operation or a swaging operation. The grooves may extend longitudinally along the elongate body and positioned diametrically opposite one another about the elongate body. The grooves facilitate improved engagement from the emitters being swaged onto the elongate body.

The lumens may be equiangularly arranged, or arranged in other suitable configurations. A proximal lead is disposed within the first lumen. The proximal lead is electrically conductive and secured to the proximal emitter. A distal lead may be disposed within the second lumen and secured to the distal emitter. The joining of the proximal and distal leads to a respective one of the proximal and distal emitters may be accomplished through a welding operation, for example, a laser welding operation. A portion of the elongate body forms an insulative spacer between the proximal and distal emitters. The proximal and distal leads extend proximally to within the hub. A thermocouple may be disposed within the third lumen. The fourth lumen may be an infusion lumen. The infusion port is defined by the elongate body and is configured to be arranged in fluid communication with the fluid coupler on the hub, and further arranged in fluid communication with the infusion module when coupled to the fluid coupler. The infusion port may be positioned on the portion of the elongate body forming the insulative spacer between the emitters.

The emitter(s) includes the inner surface, and an outer surface. The emitter is formed with slots between the inner and outer surfaces. The slots are sized such that, despite material deformation from the emitter being crimped or swaged onto the flexible distal portion of the shaft, the slots remain sufficiently defined to impart the requisite flexibility. The emitter includes a slotted portion, and end portions disposed opposite of the slotted portion. One of the leads may be secured to one of the end portions of the emitter.

The slots may be formed through a laser cutting operation or another suitable manufacturing process. The parameters of the slots may include kerf, slot pitch, cut angle, and uncut angle. The kerf may be within the range of approximately 0.015 to 0.035 mm, and more particularly approximately 0.025 mm. The slot pitch may be within a range of approximately 0.150 to 0.400 millimeters, and more particularly within the range of 0.165 to 0.215 mm, and even more particularly approximately 0.191 mm. The slot pitch may be regular or irregular. The cut angle may be within a range of approximately 50 to 100 degrees, more particularly within the range of approximately 62 to 88 degrees, and even more particularly approximately 82 degrees. The uncut angle may be within a range of approximately 10 to 30 degrees, and more particularly within the range of 18 to 21 degrees.

Owing to the flexibility of the electrode probe, it may follow the flexible conduit through a curve of at least 60 degrees, more particularly at least 90 degrees, and even more particularly at least 120 degrees, and/or deployed through the curve having a radius of curvature within the range of approximately 20 to 65 mm, more particularly within the range of approximately 30 to 55 mm.

According to a first aspect of the present disclosure, the electrode probe includes a hub, a shaft, and at least one emitter. The shaft extends from the hub and defines at least one lumen. The shaft includes a flexible distal portion. A lead is disposed within the lumen and configured to be arranged in electrical communication with a source of RF energy. The emitter is coupled to the lead and formed from conductive material. The emitter is swaged onto the flexible distal portion of the shaft. The emitter includes a slotted portion defining slots sized for the conductive material to be deformed to reduce a diameter of the emitter with swaging of the emitter onto the flexible distal portion of the shaft.

According to a second aspect of the present disclosure, the electrode probe includes a hub, a shaft, and at least one emitter. The shaft extends from the hub and defines at least one lumen. The shaft includes a flexible distal portion. A lead is disposed within the lumen and configured to be arranged in electrical communication with a source of RF energy. The emitter is coupled to the lead and formed from conductive material. The emitter is coupled to the flexible distal portion of the shaft. The emitter comprises a slotted portion defining slots, and end portions disposed opposite the slotted portion. The lead is secured to one of the end portions, for example, through laser welding.

According to a third aspect of the present disclosure, the electrode probe includes a hub, a shaft, and at least one emitter. The shaft extends from the hub and defines at least one lumen. The shaft includes a flexible distal portion. A lead is disposed within the lumen and configured to be arranged in electrical communication with a source of RF energy. The emitter is coupled to the lead and formed from conductive material. The emitter has a length of at least eight millimeters and is coupled to the flexible distal portion of the shaft. The emitter includes a slotted portion defining slots. The slotted portion has a slot pitch defined as a distance between longitudinally adjacent slots with the slot pitch being within a range of 0.150 to 0.400 millimeters.

According to a fourth aspect of the present disclosure, the electrode probe includes a hub, a shaft, and at least one emitter. The shaft extends from the hub and defines at least one lumen. The shaft includes a flexible distal portion. A lead is disposed within the lumen and configured to be arranged in electrical communication with a source of RF energy. The emitter is coupled to the lead and formed from conductive material. The emitter has a length of at least eight millimeters and is coupled to the flexible distal portion of the shaft. The emitter includes a slotted portion defining slots circumferentially disposed about an outer surface of the emitter. A cut angle is defined between opposing ends of each of the slots, and wherein the cut angle may be within a range of 50 to 90 degrees. An uncut angle is defined between a respective one of the opposing ends of axially adjacent slots. The uncut angle may be within a range of 10 to 30 degrees.

According to a fifth aspect of the present disclosure, the electrode probe includes a hub, a shaft defining at least two lumens, two leads, and two emitters. A first lead is disposed within a first of the lumens and configured to be arranged in electrical communication with a source of RF energy. A second lead is disposed within a second of the lumens and configured to be arranged in electrical communication with the source of RF energy. A distal emitter is coupled to the first lead and formed from conductive material. A proximal emitter is coupled to the first lead and formed from conductive material. The proximal and distal emitters are coupled to the flexible distal portion of the shaft. Each of the proximal and distal emitters comprise a slotted portion defining slots. A section of the flexible distal portion between the proximal and distal emitters is an insulative spacer.

Certain methods of manufacturing of the electrode probe are herein disclosed. A polymer may be extruded to include the outer surface and at least one of the lumens. The emitter is swaged onto the outer surface with the swaging operation in which the slots are narrowed, and the diameter of the emitter is reduced. In certain implementations, the swaging operation further includes swaging the emitter to be sub-flush such that the flexible distal portion is at least partially compressed. The lead(s) are positioned within the lumen of the flexible distal portion, and secured in electrical communication with the emitter(s) with the welding operation. The shaft is secured to the hub.

### BRIEF DESCRIPTION OF THE DRAWINGS

FIG. 1 is a perspective view of an ablation system including a console, a channel splitter, electrode probes for radiofrequency ablation, and infusion modules.

FIG. 2 is an elevation view of an implementation of the electrode probe.

FIG. 3 is a partial elevation view of a shaft of the electrode probe.

FIG. 4 is a partial prospective view of the distal portion of the shaft of the electrode probe.

FIG. 5 is a sectional view of the electrode probe of FIG. 4 taken along lines 5-5.

FIG. 6 is a sectional view of the electrode probe of FIG. 4 taken along lines 6-6.

FIG. 7 is a sectional view of the electrode probe of FIG. 5 taken along lines 7-7.

FIG. 8 is a perspective view of an emitter.

FIG. 9 is an elevation view of the emitter.

FIG. 10 is a detailed view of the emitter of FIG. 9 within circle 10-10.

FIG. 11 is a representation of the electrode probe being deployed within a vertebral body for ablation of a bone tumor.

FIG. 12 is a representation of the electrode probe being deployed within the vertebral body for ablation of the basivertebral nerve.

### DETAILED DESCRIPTION

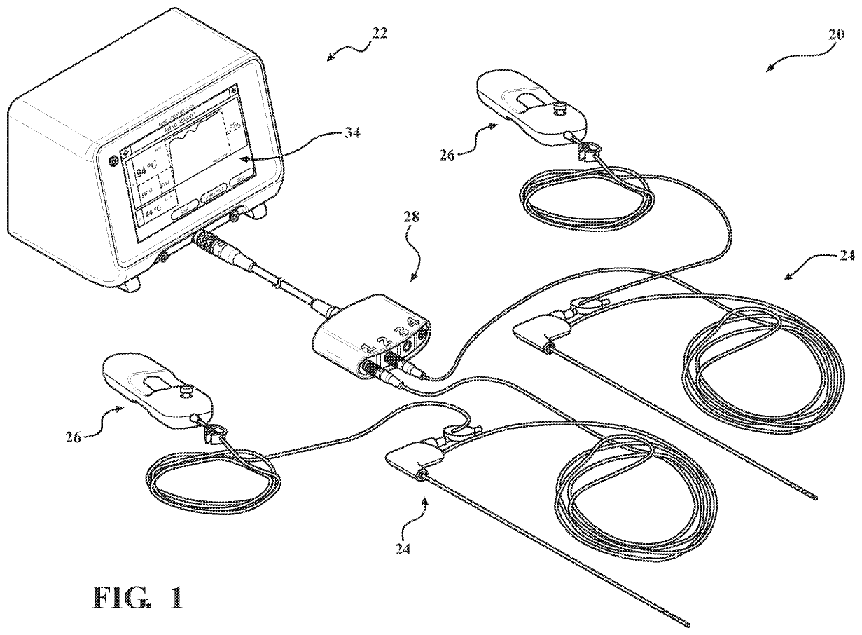

FIG. 1 shows an ablation system 20 for radiofrequency (RF) ablation of tissue. The ablation system 20 includes a console 22, at least one electrode probe 24, and optionally at least one infusion module 26. For procedures in which more than one probe 24 may be used simultaneously, the ablation system 20 may further include a cable accessory 28 to which each of the probes 24 may be individually and removably coupled. The electrode probe 24 to be described is a self-grounding bipolar electrode probe in which a proximal emitter 30 is electrically insulated from a distal emitter 32 and configured to pass RF energy therebetween to heat and ablate tissue. Alternatively, aspects the present disclosure may be provided on a monopolar electrode assembly including a single emitter for interfacing with a grounding source, for example, a ground pad.

The console 22 may include a display 34 providing a user interface. The console 22 includes a source of RF energy, for example, an RF generator. One suitable console is sold under the tradenames MultiGen (MG1), MultiGen 2 (MG2), and Optablate by Stryker Corporation (Kalamazoo, Mich.), and those described in commonly-owned International Publication No. WO 2018/0200254, published Nov. 1, 2018, International Publication No. WO 2020/0198150, published Nov. 5, 2020, and International Application No. PCT/US 2022/038635, filed Jul. 28, 2022, 2022, the entire contents of each being hereby incorporated by reference.

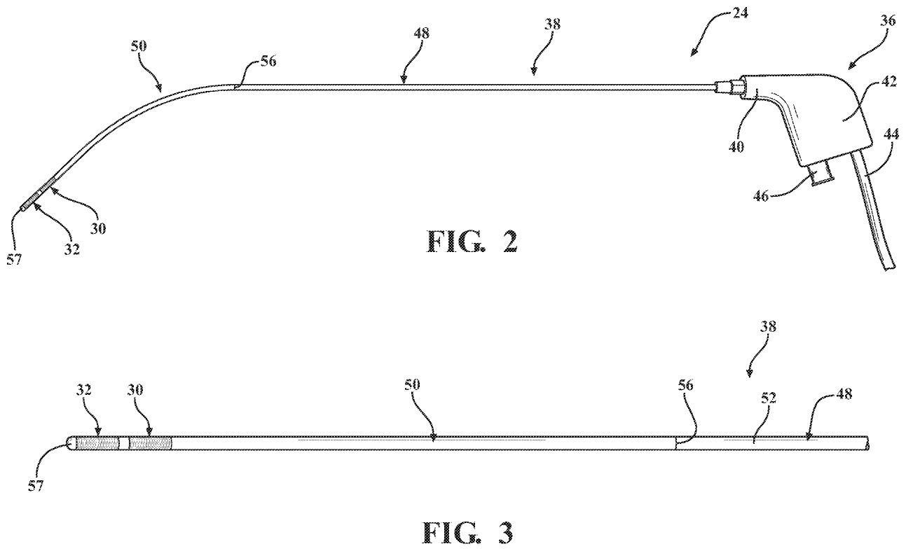

Referring now to FIGS. 2 and 3, the electrode probe 24 includes a handle or hub 36, and a shaft 38 coupled to and extending from the hub 36. The hub 36 may be sized and shaped to be ergonomically manipulated by the surgeon. For example, the hub 36 may include a neck 40 oriented along a longitudinal axis of the shaft 38, and a body 42 formed with the neck 40. The body 42 may be oriented at an angle to be downwardly and proximally sloped from the neck 40, and sized to be pinched between in index and middle fingers and the thumb of the surgeon. The shaft 38 may extend from the neck 40 of the hub 36. The hub 36 may include a power coupler configured to be removably coupled with a power line 44, or the power line 44 may extend from the body 42. The power line 44 may also transmit data between the console 22 and the electrode probe 24. Likewise, the hub 36 includes a fluid coupler 46 configured to be arranged in fluid communication with the infusion module 26.

The shaft 38 may include a flexible elongate body 54 forming at least a flexible distal portion 50 of the shaft 38. The shaft 38 may also include a proximal portion 48, which may be flexible or rigid. In the illustrated implementation, the proximal portion 48 includes a rigid sleeve 52 coaxially overlying a portion of the elongate body 54. For example, the rigid sleeve 52 is a hypotube with the elongate body 54 extending from the hypotube. In such an arrangement, the proximal portion 48 of the shaft 38 is defined between the hub 36 and a distal end 56 of the rigid sleeve 52, and the flexible distal portion 50 of the shaft 38 is defined between the distal end 56 of the rigid sleeve 52 and a distal end 57 of the elongate body 54.

The elongate body 54 may be polymeric, in other words, at least partially formed from a polymer. The elongate body 54 is preferably a tube extruded from a thermoplastic elastomer such as polyether block amide, for example, PEBAX 6333. Other suitable materials may include polyether ether ketone (PEEK), polytetrafluoroethylene (Teflon™), phenolic, polycarbonate, polysulfane, and polyoxymethylene, among others. The polymer may have a Young's modulus of less than three gigapascals (GPa). Alternatively, the elongate body 54 may be molded or shaped through other suitable manufacturing techniques, and may be formed from films, fibers, fabrics, and powders. The extrusion of the elongate body 54 is particularly well-suited for providing one or more lumens 58, 60, 62, 64 and/or one or more grooves 66, 68 to be described.

As mentioned, conventional electrode probes, especially those with fluid infusion or internal cooling, are generally incapable of achieving more than minimal curvature. The electrode probe 24 of the present disclosure advantageously includes the flexible distal portion 50 extending to the distal end 57 of the electrode probe 24. In other words, the elongate body 54 may be unitary in construction from the flexible polymer with the proximal emitter 30 and the distal emitter 32 being coupled to the elongate body 54. For conventional electrode probes that may be somewhat flexible, such as those use with cardiac ablation, the devices cannot achieve sufficient radii of curvature for posterior access within a vertebral body, among other procedures requiring sharper curved access. The insufficient flexibility of the conventional electrode probes is, in part, due to the rigidity of the emitters themselves. Stated differently, the emitters are formed from conductive material, typically metal, and therefore the rigidity associated with the metal emitters prevents the conventional electrode probe from achieving greater radii of curvature. The electrode probe 24 of the present disclosure advantageously overcomes this shortcoming by the proximal emitter 30 and the distal emitter 32 being formed with slots 70. The slots 70 are formed in a manner to impart flexibility to the emitters 30, 32 themselves. Therefore, the emitters 30, 32 are flexible in addition to the flexibility of the elongate body 54, and consequently the electrode probe 24 is capable of achieving higher radii of curvature for tighter turns, such as within the vertebral body once deployed beyond an access cannula 100.

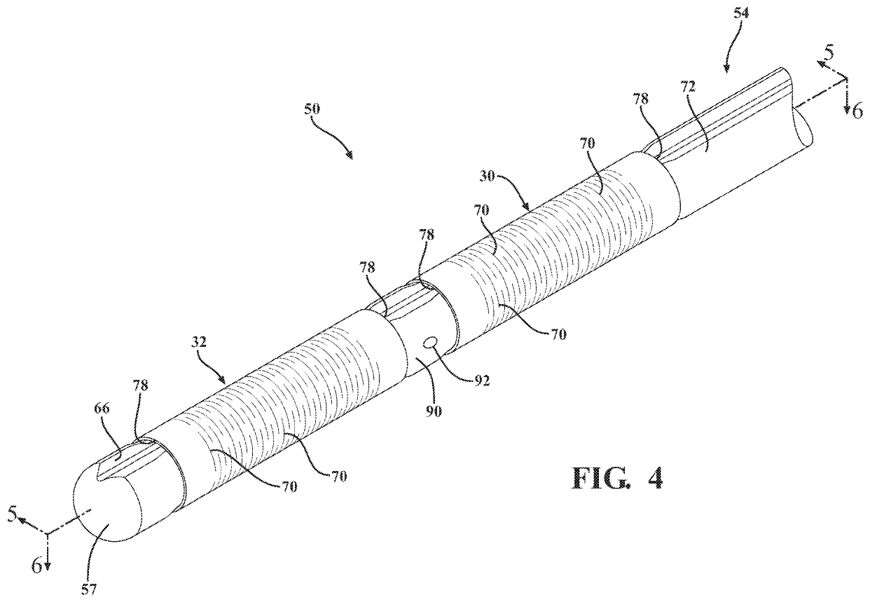

FIG. 4 shows in greater detail the proximal and distal emitters 30, 32 coupled to the flexible distal portion 50 of the elongate body 54. With the flexible distal portion 50 being polymeric, the emitters 30, 32 may be secured to the flexible distal portion 50 through a crimping operation, or preferably a swaging operation. The flexible distal portion 50 deforms with the swaging operation from compressive forces on an outer surface 72 of the elongate body 54 from an inner surface 74 of the emitters 30, 32. Grooves 66, 68 may extend longitudinally along the elongate body 54 and positioned diametrically opposite one another about the elongate body 54. Alternatively, it is contemplated that more or less grooves may be provided, and radially arranged any suitable configuration. The grooves 66, 68 are configured to facilitate improved engagement from the emitters 30, 32 being swaged onto the elongate body 54. More particularly, during the swaging operation, ridges adjacent the grooves 66, 68 may deform towards or into the groove 66, 68 to provide a secure friction fit or interference fit in which the emitters 30, 32 are flush or sub-flush with the outer surface 72 of the elongate body 54. FIG. 4 generally shows the deformation associated with the swaging operation by deformation of the elongate body 54 adjacent to ends 78 of the emitters 30, 32.

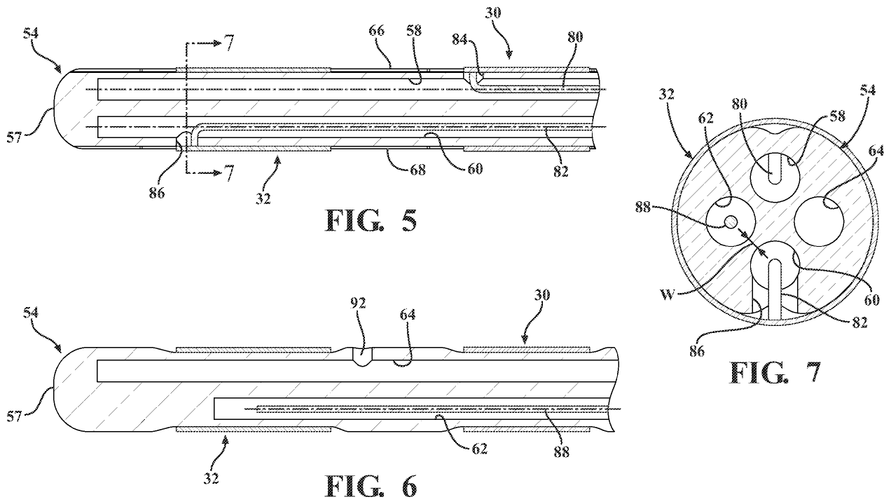

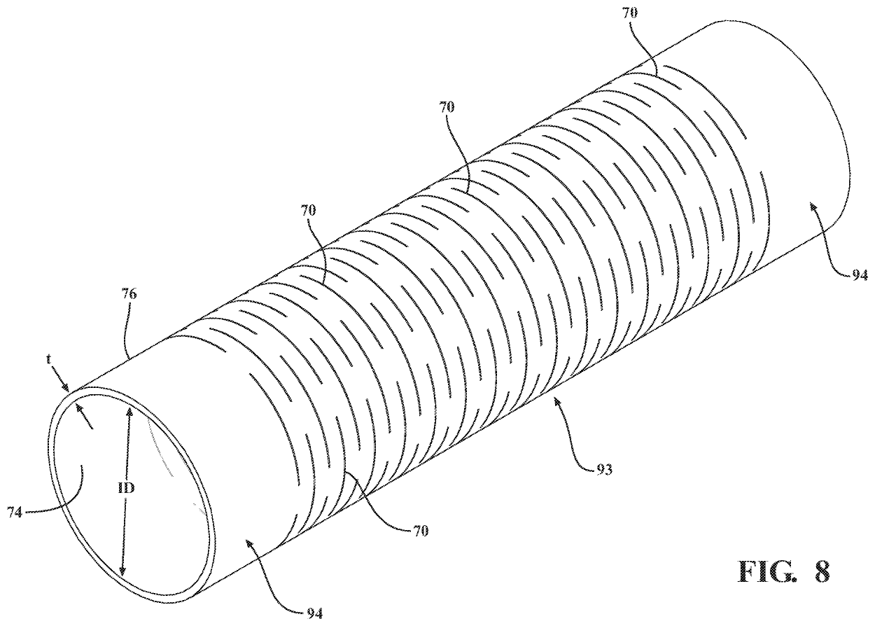

The sectional views of FIGS. 5-7 illustrate several internal structures and components of the electrode probe 24. The extrusion of the elongate body 54 may include at least one lumen. The exemplary implementation includes a first lumen 58, a second lumen 60, a third lumen 62, and a fourth lumen 64. The lumens 58, 60, 62, 64 may be equiangularly arranged as shown in the axial section view of FIG. 7, but other positional configurations are contemplated. The lumens 58, 60, 62, 64 may have same or different diameters, and the angular positioning may be based on the number or size of the lumens 58, 60, 62, 64. The lumens 58, 60, 62, 64 may extend longitudinally parallel to one another within the elongate body 54 and not be in fluid communication with one another.

A proximal lead 80 is disposed within the first lumen 58. The proximal lead 80 is electrically conductive and secured to the proximal emitter 30. More particularly, the proximal lead 80 extends through the first lumen 58, passes through an aperture 84 defined by the elongate body 54, and joined to the inner side 74 of the proximal emitter 30. Likewise, a distal lead 82 may be disposed within the second lumen 60 and secured to the distal emitter 32. The distal lead 82 extends through the second lumen 60, passes through another aperture 86 defined by the elongate body 54, and joined to the inner side 74 of the distal emitter 32. The joining of the proximal and distal leads 80, 82 to a respective one of the proximal and distal emitters 30, 32 may be accomplished through a welding operation, for example, a laser welding operation.

The proximal and distal leads 80, 82 extend proximally to within the hub 36. The proximal and distal leads 80, 82 are configured to be arranged in electrical communication with the source of RF energy via the power line 44. The RF energy supplied to the emitters 30, 32 via the proximal and distal leads 80, 82 generates an RF pathway and consequently an ablation zone when applied adjacent to target tissue within the anatomy. To that end, the emitters 30, 32 are axially spaced apart from one another and of opposite polarity. A portion of the elongate body 54 forms an insulative spacer 90 between the proximal and distal emitters 30, 32. For example, in implementations where the elongate body 54 is formed from PEBAX, the elongate body 54 itself is non-conductive and therefore forms the insulative spacer 90. The emitters 30, 32 are therefore electrically insulated without the need for a discrete insulative spacer that may require mechanical coupling along with adhesives, threading, lap joints, or the like. In addition to increased flexibility and reduced manufacturing complexity and cost, the arrangement eliminates interfaces associated with risk of egress of infusion fluid, particularly with bending of the electrode probe 24 at greater bend angles and sharper curvatures.

A thermocouple 88 may be disposed within the third lumen 62. The thermocouple 88 may be secured to the elongate body 54 within the third lumen 62, for example, with adhesive, an internal cap, or other joining means. Alternatively, a distal end of the thermocouple 88 may be embedded within the elongate body 54. The thermocouple 88 extends to within the hub 36 and is configured to be arranged in electrical communication with the console 22. The thermocouple 88 is configured to sense a temperature indicative of the extent of heating of the target tissue. The console 22 may adjust parameters of the ablation procedure, such as the amount of RF energy being delivered, based on the temperature sensed by the thermocouple 88.

The fourth lumen 64 may be an infusion lumen. The elongate body 54 may define an infusion port 92 in fluid communication with the fourth lumen 64. The infusion port 92 is defined by the elongate body 54 and is configured to be arranged in fluid communication with the fluid coupler 46 on the hub 36, and further arranged in fluid communication with the infusion module 26 when coupled to the fluid coupler 46. The illustrated implementation shows the infusion port 92 positioned on the portion of the elongate body 54 forming the insulative spacer 90 between the emitters 30, 32. Other positions for the infusion port 92 are contemplated, such as proximal to the proximal emitter 30, and/or at the distal end 57 of the electrode probe 24. More than one infusion port may be provided.

The multi-lumen arrangement prevents potential compromise of electrical components with the infusion fluid. Further, since the elongate body 54 itself provides the barrier separating the lumens 58, 60, 62, 64, there is little sacrifice to the flexibility of the elongate body 54 and lesser concern for compromise of internal subcomponents or interfaces between the same. As mentioned, the extrusion of the elongate body 54 provides for intricate internal geometries (i.e., the lumens 58, 60, 62, 64) without significant manufacturing complexities. This is particularly relevant given the dimensions and tolerances of the elongate body 54 and its geometries. For example, the elongate body 54 may have an outer diameter within the range of approximately 1.75 to 2.25 millimeters (mm), and more particularly within the range of approximately 1.90 to 2.00 mm. The lumens 58, 60, 62, 64 may have the same or different inner diameters with an exemplary inner diameter being within the range of approximately 0.40 to 0.60 mm, and more particularly within the range of approximately 0.45 to 0.50 mm. Further, a thickness of the wall (w) defined between adjacent pairs of the lumens 58, 60, 62, 64 (see FIG. 7) may be within the range of approximately 0.10 to 0.15 mm, and more particularly approximately 0.125 mm. The apertures 84, 86 and/or the infusion port 92 may have a diameter within the range of approximately 0.40 to 0.5 mm, and more particularly approximately 0.45 mm. The apertures 84, 86 may be spaced apart from one another by a distance within the range of approximately 5.0 to 8.0 mm, and more particularly within the range of approximately 6 to 7 mm. The distal ends of the lumens 58, 60, 62, 64 may terminate prior to the distal end 57 of the electrode probe 24, leaving a tipped region of approximately one millimeter. Lastly, the grooves 66, 68 may be formed with a radius of approximately 0.1 mm. Such geometries and tolerances may not be feasible with other manufacturing techniques in a cost-effective manner.

FIG. 8 is a perspective view of an emitter, for example, the proximal emitter 30 or the distal emitter 32. The emitters 30, 32 may be the same or different, and are describe hereto forward in the singular. The emitter 30, 32 includes the inner surface 74, and an outer surface 76 opposite the inner surface 74. A thickness (t) of the emitter 30, 32 is defined between the inner and outer surfaces 74, 76, and an inner diameter (ID) of the emitter 30, 32 is defined by the inner surface 74. The thickness may be within the range of approximately 0.05 to 0.10 mm, and more particularly within the range of approximately 0.07 to 0.08 mm. The inner diameter may be sized for the elongate body 54 to pass through the emitter 30, 32 during assembly with less than one pound of insertion force. An exemplary range of the inner diameter is approximately 1.8 to 2.1 mm, and more particularly approximately 1.9 to 2.0 mm.

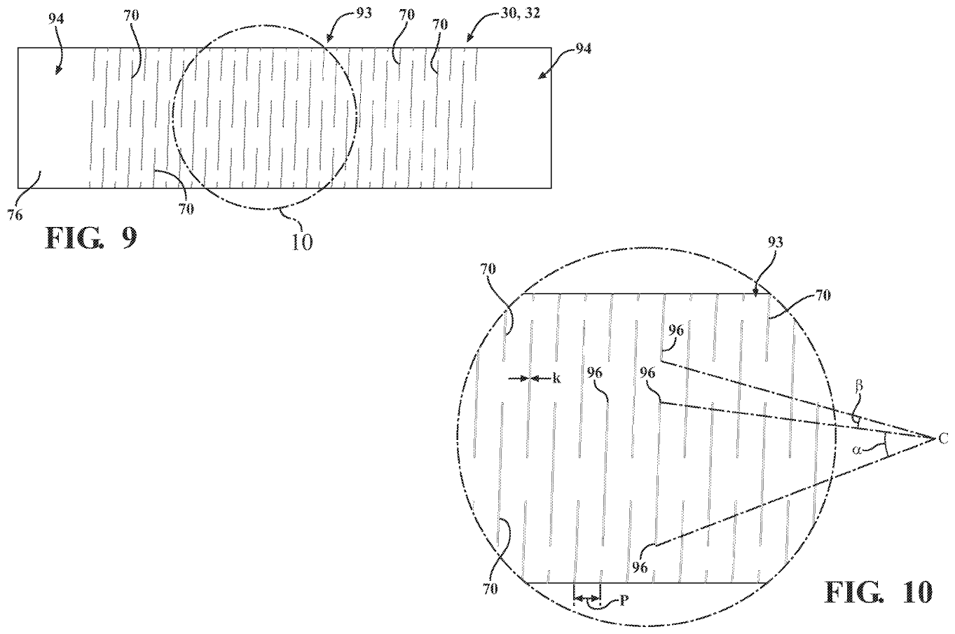

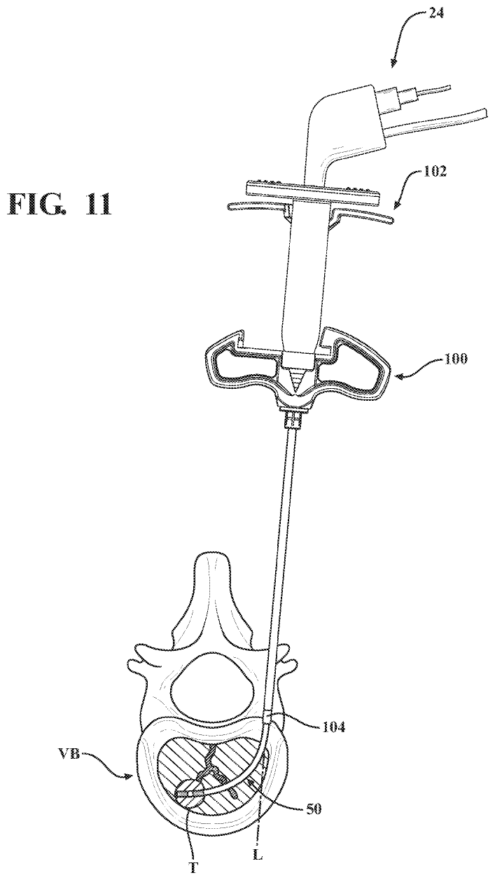

As mentioned, the emitter 30, 32 is formed with slots 70 to preserve flexibility. More particularly, a length of the emitter 30, 32 may be at least six millimeters, and such appreciable lengths otherwise devoid of the slots 70 may not achieve the requisite flexibility for certain clinical applications. Owing to the benefits of the slots 70, the length of the emitter 30, 32 may be at least eight, ten, or twelve or more millimeters. The emitter 30, 32 may be relatively longer in implementations in which the electrode probe 24 is monopolar. Further, as to be described, the slots 70 are sized such that, despite material deformation from the emitter 30, 32 being crimped or swaged onto the flexible distal portion 50 of the shaft 38, the slots 70 remain sufficiently defined to impart the requisite flexibility. With concurrent reference to FIGS. 9 and 10, the emitter 30, 32 includes a slotted portion 93, and end portions 94 disposed opposite of the slotted portion 93. The slotted portion 93 may assume 60, 70, 80 or more percent of the length of the emitter 30, 32. For example, the end portions 94 may each have a length within the range of approximately 0.90 to 1.10 mm. In certain implementations, one of the leads 80, 82 is secured to one of the end portions 94 of the emitter 30, 32. For example, the lead 80, 82 may be laser welded to a distal one of the end portions 94, as shown in FIG. 5.

The slots 70 may be formed through a laser cutting operation or another suitable manufacturing process. The slots 70 may be formed with tuned parameters to impart the desired flexibility while permitting the emitter 30, 32 to be secured to the flexible distal portion 50 with the swaging operation. With reference to FIG. 10, the parameters may include kerf (k), slot pitch (P), cut angle (α), and uncut angle (β). The kerf may be defined as a width or size of each slot 70 prior to the swaging operation. The kerf may be within the range of approximately 0.015 to 0.035 mm, and more particularly approximately 0.025 mm.

The kerf is sized for the slots 70 to be narrowed during the swaging operation with little sacrifice of flexibility of the emitter 30, 32. More particularly, the swaging operation may be rotary swaging, roller swaging, or radial forging in which dies are used to decrease the inner and outer diameters of the emitter 30, 32 onto the elongate body 54. The slots 70 provide the clearance necessary for the deformation of the conductive material without producing “fins” between the dies that are often associated with certain operations such as crimping. As a result, after the swaging operation, the outer surface 76 of the emitter 30, 32 is at least flush (or sub-flush) with an outer surface 72 of the elongate body 54, and characterized by a smooth outer contour. While the slots 70 narrow during the deformation to, for example, about 0.01 mm, the slots 70 remain sufficiently sized to impart the flexibility to the flexible distal portion 50. It is contemplated that the slots 70 may have the same or different kerf. For example, the slots 70 nearer to a center of the slotted portion 93 may be wider or narrower than the slots 70 nearer to the end portions 94.

With continued reference to FIG. 10, the slot pitch of the slots 70 may be defined as a distance between longitudinally adjacent slots 70. In other words, for every revolution of the laser cutter about the emitter 30, 32, the laser cutter has moved axially by the slot pitch. As appreciated from the slight tilt when viewed in elevation, the laser cutting operation may generate a spiral-like pattern along the length of the emitter 30, 32. In an exemplary implementation, the slot pitch is within a range of approximately 0.150 to 0.400 millimeters, and more particularly within the range of 0.165 to 0.215 mm, and even more particularly approximately 0.191 mm. The slot pitch may be regular or irregular such that the slots 70 may be uniformly axially spaced or differently spaced along the length of the emitter 30, 32.

The cut angle (α) of the slots 70 may be defined between opposing ends 96 of each of the slots 70. The uncut angle (β) of the slots 70 may be defined between a respective one of the opposing ends 96 of axially adjacent slots 70. The cut angle and the uncut angle are defined relative to a coaxial center (C) of the emitter 30, 32 with FIG. 10 annotated for illustrative purposes. The cut angle and uncut angle, from a practical standpoint, characterize the arcs subtended by the slots 70 and portions of the emitter 30, 32 between the “next” circumferential slot in the laser cutting operation. In other words, the laser cutting operation includes cutting the slot 70, pausing as the laser cutter moves about the emitter 30, 32 (or the emitter 30, 32 is rotated and advanced), then again cutting the next circumferential slot 70. The cut angle may be within a range of approximately 50 to 100 degrees, more particularly within the range of approximately 62 to 88 degrees, and even more particularly approximately 82 degrees. The uncut angle may be within a range of approximately 10 to 30 degrees, and more particularly within the range of 18 to 21 degrees.

In view of the foregoing characteristics of the electrode probe 24, inventive methods of manufacturing of the electrode probe 24 are hereby provided. In particular, the method may include extruding a polymer to include the outer surface 72 and at least one of the lumens 58, 60, 62, 64. The polymer forms the flexible distal portion 50 of the shaft 38. The emitter 30, 32 formed from conductive material is provided, and the slots 70 are formed through the conductive material with the laser cutting operation. The emitter 30, 32 is positioned over the outer surface 72 of the flexible distal portion 50. The emitter 30, 32 is swaged onto the outer surface 72 with the swaging operation in which the slots 70 are narrowed and the diameter of the emitter 30, 32 is reduced. The lead 80, 82 is positioned within the lumen of the flexible distal portion 50, and secured in electrical communication with the emitter 30, 32 with the welding operation. The shaft 38 is secured to the hub 36. Certain counterpart steps may be repeated for implementations in which there is a second emitter 30, 32.

In certain implementations, the laser cutting operation further includes advancing the emitter 30, 32 by a fixed distance for each revolution of the laser cutter about the emitter 30, 32 to define the slot pitch. The slot pitch may be within a range of 0.150 to 0.400 mm, or more particularly 0.191 mm. The laser cutting operation may include laser cutting the slots 70 to include opposing ends 96 defining the cut angle within a range of 50 to 90 degrees, or more particularly 82 degrees. The laser cutting operation may further include not laser cutting portions of the emitter 30, 32 between the opposing ends 96 of axially adjacent slots 70 to define the uncut angle within a range of 10 to 30 degrees, or more particularly approximately 21 degrees. The steps of cutting and not cutting may be alternated and repeated as the emitter 30, 32 is advanced during the laser cutting operation.

In certain implementations, the swaging operation further includes swaging the emitter 30, 32 to be sub-flush such that the flexible distal portion 50 is at least partially compressed. In other words, the outer diameter of the emitter 30, 32 may be less than the outer diameter of the elongate body 54 adjacent to the emitter 30, 32. A feed rate for the swaging operating may be set at within the range of 0.5 to 5.0 millimeters per second. The swaging operating may be performed at 55 Hertz. The swaging operation may also narrow the slots 70 to a kerf of about 0.01 millimeters. In certain implementations, the method includes pre-crimping the emitter 30, 32 after the step of positioning the emitter 30, 32 over the outer surface 72 of the flexible distal portion 50.

In certain implementations, the conductive material of the emitter 30, 32 may be annealed. The step of annealing may be performed to soften the conductive material prior to the step of swaging, and to improve radiopacity of the emitter 30, 32. Alternatively, the emitter 30, 32 may be formed from stainless steel, and more particularly fully-hardened stainless steel. The method may include electroplating the emitter 30, 32 with a radiopaque material prior to the step of swaging the emitter 30, 32 onto the outer surface 72 of the flexible distal portion 50. The radiopaque material may be gold or platinum iridium, but other radiopaque materials are contemplated. Additional radiopaque elements may be included at suitable positions to aid in placement of the electrode probe 24 under fluoroscopic guidance.

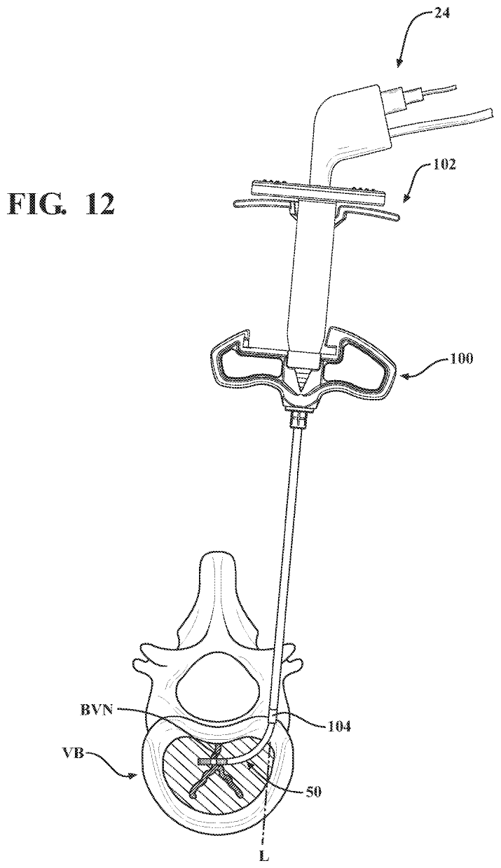

The electrode probe 24 of the present disclosure facilitates the treatment of tissue in anatomical locations not previously accessible with conventional devices. More particularly, the flexibility of the elongate body 54 and the emitter(s) 30, 32 provides access to the posterior portion of the vertebral body by achieving greater degrees of curvature and/or sharper radii of curvature. Referring now to FIGS. 11 and 12, the access cannula 100 may be directed through the pedicle to provide access within the vertebral body (VB). An introducer device including a conduit assembly 102 may be deployed offset from a longitudinal axis (L) of the access cannula 100. A suitable introducer device is disclosed in commonly-owned U.S. Pat. No. 9,839,443, issued Dec. 12, 2017, and commonly-owned United States Publication No. 2022/066743, published Mar. 31, 2022, the entire contents of each being incorporated by reference. The conduit assembly 102, including a flexible conduit 104, remains curved through cancellous bone within the vertebral body.

The electrode probe 24 is directed through the flexible conduit 104. Owing to the flexibility of the electrode probe 24, it may follow the flexible conduit 104 through a curve of at least 60 degrees, more particularly at least 90 degrees, and even more particularly at least 120 degrees. Further, the electrode probe 24 has sufficient flexibility to be deployed through the curve having a radius of curvature within the range of approximately 20 to 65 mm, more particularly within the range of approximately 30 to 55 mm.

The source of RF energy of the console 22 is operated to ablate the target tissue. FIG. 11 shows the electrode probe 24 deployed to ablate a bone tumor (T) contralateral from the pedicle through which the access cannula 14 is directed. FIG. 12 shows the electrode probe 24 being deployed markedly posterior to access the trunk of the basivertebral nerve (BVN). It is appreciated that the ablation system 20 of the present disclosure may be used at any suitable anatomical location, including osseous and non-osseous applications. Exemplary non-osseous applications include facet rhizotomy, sacroiliac nerve block, genicular nerve block, and the like.

Certain inventive aspects of the present disclosure are appreciated with reference to the following exemplary clauses.

Clause 1—A method of manufacturing an electrode probe for radiofrequency ablation, the method comprising: extruding a polymer to include an outer surface and a lumen, wherein the polymer forms a flexible distal portion of a shaft of the electrode probe; providing an emitter formed from conductive material; forming slots through the conductive material with a laser cutting operation; positioning the emitter over the outer surface of the flexible distal portion; swaging the emitter onto the outer surface with a swaging operation in which the slots are narrowed and a diameter of the emitter is reduced; positioning a lead within the lumen of the flexible distal portion; securing the lead in electrical communication with the emitter with a welding operation; and securing the shaft to a hub.

Clause 2—The method of clause 1, wherein the slots are laser cut to have a width within a range of 0.020 to 0.030 millimeters.

Clause 3—The method of clause 1 or 2, wherein the laser cutting operation further comprises advancing the emitter by a fixed distance for each revolution of laser cutting about the emitter to define a slot pitch, wherein the slot pitch is within a range of 0.150 to 0.400 millimeters.

Clause 4—The method of clause 3, wherein the slot pitch is about 0.191 millimeters.

Clause 5—The method of any one of clauses 1-4, wherein the laser cutting operation further comprises laser cutting the slots to include opposing ends defining a cut angle, wherein the cut angle is within a range of 50 to 90 degrees.

Clause 6—The method of clause 5, wherein the cut angle is 82 degrees.

Clause 7—The method of clause 5 or 6, wherein the laser cutting operation further comprises not laser cutting portions of the emitter between the opposing ends of axially adjacent slots to define an uncut angle, wherein the uncut angle is within a range of 10 to 30 degrees.

Clause 8—The method of clause 7, wherein the uncut angle is 21 degrees.

Clause 9—The method of clause 7 or 8, further comprising alternating and repeating the steps of laser cutting the slots and not laser cutting the portions as the emitter is advanced during the laser cutting operation.

Clause 10—The method of any one of clauses 1-9, further comprising electroplating the emitter with a radiopaque material prior to the step of swaging the emitter onto the outer surface of the flexible distal portion.

Clause 11—The method of clause 10, wherein the radiopaque material is gold or platinum iridium.

Clause 12—The method of any one of clauses 1-9, further comprising annealing the conductive material of the emitter.

Clause 13—The method of any one of clauses 1-12, further comprising pre-crimping the emitter after to the step of positioning the emitter over the outer surface of the flexible distal portion.

Clause 14—The method of any one of clauses 1-13, wherein the swaging operating further comprises setting a feed rate of the emitter at 0.5 to 5.0 millimeters per second.

Clause 15—The method of any one of clauses 25-38, wherein the swaging operating is performed at approximately 55 Hertz.

Clause 16—The method of any one of clauses 1-14, wherein the swaging operation further comprises swaging the emitter to be sub-flush such that the flexible distal portion is at least partially compressed.

Clause 17—The method of any one of clauses 1-16, wherein the swaging operation narrows the slots to a kerf of about 0.01 millimeters.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. An electrode probe for radiofrequency ablation, the electrode probe comprising:
a hub;
a shaft extending from the hub, defining at least one lumen, and comprising a flexible distal portion;
a lead disposed within the lumen and configured to be arranged in electrical communication with a source of RF energy; and
an emitter coupled to the lead and formed from conductive material, wherein the emitter is swaged onto the flexible distal portion of the shaft, wherein the emitter comprises a slotted portion defining slots, wherein the slots are narrowed by the conductive material being deformed with the swaging of the emitter onto the flexible distal portion of the shaft.

2. The electrode probe of claim 1, wherein the emitter further comprises end portions disposed opposite the slotted portion, and wherein the lead is welded to one of the end portions.

3. The electrode probe of claim 2, wherein the lead is laser welded to a distal one of the end portions.

4. The electrode probe of claim 1, wherein a thickness of the emitter defined between an outer diameter and an inner diameter is within range of 0.05 to 0.10 millimeters.

5. The electrode probe of claim 1, wherein a length of the emitter is at least eight millimeters.

6. An electrode probe for radiofrequency ablation, the electrode probe comprising:
a hub;
a shaft extending from the hub, defining a lumen, and comprising flexible distal portion;
a lead disposed within the lumen and configured to be arranged in electrical communication with a source of RF energy; and
an emitter coupled to the lead and formed from a conductive material, wherein the emitter is coupled to the flexible distal portion of the shaft,
wherein the emitter comprises a slotted portion defining slots, and end portions disposed opposite the slotted portion, and wherein the lead is secured to one of the end portions.

7. The electrode probe of claim 6, wherein the lead is laser welded to a distal one of the end portions.

8-13. (canceled)

14. The electrode probe of claim 1, wherein a width of the slots are within a range of 0.020 to 0.030 millimeters prior to swaging the emitter on the flexible distal portion.

15. The electrode probe of claim 1, wherein the emitter is hardened steel, and wherein the emitter is electroplated with one of gold and platinum iridium.

16. The electrode probe of claim 1, wherein the conductive material is annealed.

17. The electrode probe of claim 1, wherein the flexible distal portion defines an infusion port configured to be arranged in fluid communication with a source of infusion liquid.

18. An electrode probe for radiofrequency ablation, the electrode probe comprising:
a hub;
a shaft extending from the hub, defining lumens, and comprising a flexible distal portion;
a first lead disposed within a first of the lumens and configured to be arranged in electrical communication with a source of RF energy;
a second lead disposed within a second of the lumens and configured to be arranged in electrical communication with the source of RF energy;
a distal emitter coupled to the first lead and formed from conductive material; and
a proximal emitter coupled to the first lead and formed from conductive material, wherein the proximal and distal emitters are coupled to the flexible distal portion of the shaft,
wherein each of the proximal and distal emitters comprise a slotted portion defining slots, and wherein a section of the flexible distal portion between the proximal and distal emitters is an insulative spacer.

19. The electrode probe of claim 18, further comprising a thermocouple disposed within a third of the lumens.

20. The electrode probe of claim 18, or wherein the section of the flexible distal portion defines an infusion port in fluid communication with a fourth of the lumens.

21. The electrode probe of claim 18, wherein each of the proximal emitter and the distal emitter is swaged onto the flexible distal portion of the shaft.

22. The electrode probe of claim 1, wherein the shaft further comprises a rigid sleeve disposed over a proximal portion of the shaft from which the flexible distal portion extends, and wherein the rigid sleeve is secured to the hub.

23. (canceled)

24. The electrode probe of claim 1, wherein the flexible distal portion defines at least one longitudinal groove configured to facilitate coupling of the emitter to the shaft.

25. The electrode probe of claim 1, wherein the slotted portion has a slot pitch defined as a distance between longitudinally adjacent slots with the slot pitch being within a range of 0.150 to 0.400 millimeters.

26. The electrode probe of claim 1, wherein the emitter comprises a slotted portion defining slots circumferentially disposed about an outer surface of the emitter, wherein a cut angle is defined between opposing ends of each of the slots, and wherein the cut angle is within a range of 50 to 90 degrees.

27. The electrode probe of claim 26, wherein an uncut angle is defined between a respective one of the opposing ends of axially adjacent slots, and wherein the uncut angle is within a range of 10 to 30 degrees.
